(19) United States
(12) Patent Application Publication
(10) Pub. No.: US 2022/0389367 A1
(43) Pub. Date: Dec. 8, 2022

(54) CELL CULTURE VESSELS AND MONITORING SYSTEMS FOR NON-INVASIVE CELL CULTURE MONITORING

(71) Applicant: CORNING INCORPORATED, CORNING, NY (US)

(72) Inventors: Gregory Roger Martin, Acton, ME (US); Mark Christian Sanson, Macedon, NY (US); Nikhil Baburam Vasudeo, Pune, Baner (IN); Joseph Christopher Wall, Southborough, MA (US)

(21) Appl. No.: 17/776,044

(22) PCT Filed: Oct. 26, 2020
(86) PCT No.: PCT/US2020/057308

§ 371 Date: May 11, 2022

Related U.S. Application Data
(60) Provisional application No. 62/935,215, filed on Nov. 14, 2019.
(60) Provisional application No. 62/938,687, filed on Nov. 21, 2019.

(51) Int. Cl. C12M 1/34; C12M 1/12; C12M 1/24

(52) U.S. Cl. CPC C12M 41/36; C12M 23/04; C12M 23/08; C12M 41/32

(57) ABSTRACT

A cell culture vessel, cell culture monitoring system, and Raman spectroscopy system for non-invasive measuring of a cell culture are provided. The vessel includes a cell culture chamber that operates as a closed system, a wall defining a boundary of the cell culture chamber and separating the interior space of the cell culture chamber from an exterior of the cell culture chamber, and a window disposed in the wall and separating the interior space of the cell culture chamber from an exterior of the cell culture chamber. The cell culture chamber includes an interior space for housing at least one of the cell culture and a cell culture media. The window includes a polymer and allows monitoring of the cell culture via a monitoring module disposed on the exterior without the monitoring module coming into physical contact with the cell culture or the cell culture media.

### CROSS REFERENCE TO RELATED APPLICATIONS

This application claims the benefit of priority under 35 U.S.C § 120 of U.S. Provisional Application Ser. No. 62/935,215 filed on Nov. 14, 2019 and U.S. Provisional Application Ser. No. 62/938,687 filed on Nov. 21, 2019, the contents of which are relied upon and incorporated herein by reference in their entireties.

### FIELD

The present disclosure relates generally to cell culture monitoring, and more specifically to non-invasive cell culture monitoring using cell culture vessels and cell culture monitoring systems including cell culture vessels with an optical window and Raman spectroscopy optical devices.

### BACKGROUND

Cell cultures are widely used to provide an artificial environment for cell growth. Cells may grow in suspension or adhered to a surface in a cell culture vessel. The processing of cell cultures includes two principal activities, monitoring cell growth and health (confluence and morphology) and ensuring a suitable environment for cell growth (e.g., pH, glucose, and lactate levels). Production cost of cell cultures is extremely high due to low yield, high labor cost, intensive manual workflow, and costly clean room environments where processing is often performed. Monitoring methods of cell cultures are a significant factor for increasing yield and decreasing costs.

Current methods for both viewing cells and measuring analytes can be time consuming and require direct access to the vessel, which risks breaking the sterility of the vessel's environment. Scientists often use the naked eye or microscopes to view the confluence of cells. Unfortunately, these methods require direct access to the vessel, which often slows or stops cell growth. Moreover, direct access methods make it difficult or impossible to automate the process. Cell culture processing is traditionally monitored (e.g., the presence of certain analytes) through invasive and semi-invasive methods which utilize components such as probe sensors, pipettes, draw-off tubes, or patches.

For example, the act of monitoring by drawing media from the vessel is not ideal because it may disturb the cells in multiple ways that can be detrimental to their growth process. It generally requires the vessel to be opened, which can lead to introduction of contaminants. Also, the cells can experience a decrease in temperature below the ideal temperature maintained in the incubator, which, in some cases, can have an unintended effect on the cells. Further, in the case of adherent cell culture vessels, it can cause the media to move over a surface of the vessel or growth substrate as the vessel is handled by the operator. This motion can lead to release of cells from the surface and subsequent loss of the cells during, for example, a feeding. In addition, drawing off methods require a small percentage of the media to be removed from the vessel, which alters the composition or quantities of material in the system. If frequent measurements are required, this can lead to significant media volume reductions that will require addition of media to compensate, further modifying the culture conditions of the sampled vessel compared to other vessels in the batch that are not being analyzed. This variation in conditions leads to unpredictable results and variability in results between vessels in a batch.

Another analytical technique used for monitoring involves Raman spectroscopy and the use of probes. Raman spectroscopy is an analytical technique that uses light scattering to determine identities and concentrations of various molecules in a substance by illuminating the substance with monochromatic light and then measuring the individual wavelengths and their intensities in the scattered light. This analytical tool is commonly used in chemistry to identify types and concentrations of the molecules based on their structural fingerprints, and is suitable for analyzing aqueous and other liquid environments as well as for analyzing solids, gels, gases and powders. Raman spectroscopy is a technique that is based on the vibrational and rotational modes in a system. For example, in a typical Raman spectroscopy system, a sample is illuminated with a laser that optically excites the molecules in the sample, and a small fraction of the light is elastically scattered at wavelengths that are slightly lower or higher than the excitation wavelength. This shift in frequency, if the final state is lower in energy, is called a Stokes shift and, if the scattered photon is shifted to a higher frequency, is called an anti-Stokes shift.

Raman spectroscopy systems conventionally include probes that facilitate measuring Raman spectra of samples remote from a light source and a detector. The probe is optically connected to the light source through a first optical fiber (i.e., a “pump” or “excitation” fiber) and optically connected to the detector through a second optical fiber (i.e., a “receive,” “return,” or “emission” fiber). Raman spectrometers are commercially available with probes that may be immersed into a specimen, liquid, or powder, where the Raman probe can couple the pump and receive fibers to/from the sample. Raman probes have a collection optic at the end that is placed into contact with the specimen being tested. This collection optic is typically a ball (i.e., spherical). A ball-shaped optic is easy and inexpensive to produce and can collect a relatively large amount of light compared to other single optics that are not a ball lens. However, a ball lens also collects light that is very close to the front or distal surface of the ball. In other words, the focus point of the optic is close to the front surface of the optic. This is a disadvantage when attempting to use a ball lens on the end of a probe to view a specimen through a barrier when trying not to disturb or contaminate the subject being observed, such as trying to analyze a component through the wall of a cell culture vessel that is a closed system.

Because conventional cell culture methods require some type of contact with the invasive or semi-invasive components within the cell growth environment, the systems cannot operate as closed systems. Thus, there is a need for improved cell culture monitoring devices, systems, and methods for non-invasive monitoring of closed systems. Such non-invasive monitoring of closed systems may allow for the use of automation to better control cell culture media compositions and cell growth and health, and may allow sterility to be maintained throughout growth, thereby reducing the requirements and cost of a clean room.

### SUMMARY

According to embodiments of this disclosure, a cell culture vessel is provided that allows non-invasive measuring of a cell culture. The vessel includes a cell culture chamber that can operate as a closed-system, and a wall defining a boundary of the cell culture chamber and separating the interior space of the cell culture chamber from an exterior of the cell culture chamber. The cell culture chamber has an interior space for housing at least one of the cell culture and a cell culture media. The vessel also includes a window disposed in the wall and separating the interior space of the cell culture chamber from an exterior of the cell culture chamber. The window includes a polymer and allows monitoring of the cell culture via a monitoring module disposed on the exterior without the monitoring module coming into physical contact with the cell culture or the cell culture media.

According to some embodiments, the vessel further includes a monitoring module disposed on the exterior side of the window to monitor an aspect of the cell culture through the window. The monitoring module can include an analyte monitor, which can include a spectral element that can emit one or more excitation wavelengths of light and capture emitted light from a media layer within the cell culture chamber. In some embodiments, the spectral element can perform Raman spectroscopy on the media layer. As an aspect of some embodiments, the analyte monitor monitors at least one of glucose, lactose, and glutamine within the cell culture chamber.

According to one or more embodiments of this disclosure, the window includes a material with an emission spectra that does not interfere with the monitoring module's detection of analytes in the cell culture vessel. In some embodiments, the window includes a silicone material, such as polydimethylsiloxane. The window may include a polymer that has no carbon-carbon covalent bonds.

According to embodiments of this disclosure, a cell culture monitoring system for non-invasive monitoring of a cell culture is provided. The system includes a cell culture vessel having a cell culture chamber arranged to operate as a closed system, the cell culture chamber having an interior space for housing at least one of the cell culture and a cell culture media. The system further includes a wall defining a boundary of the cell culture chamber and separating the interior space of the cell culture chamber from an exterior of the cell culture chamber; and a monitoring system disposed on the exterior of the cell culture chamber to analyze the cell culture or the cell culture media in the interior space by delivering light through an optical element and into the interior space, wherein the light is transmitted into the optical element through a first surface of the optical element and out of the optical element through a second surface of the optical element, at least one of the first surface and the second surface having an aspheric shape.

According to one or more embodiments, a Raman spectroscopy system is provided that is configured for non-invasive monitoring of a specimen. The system includes a probe having a fiber optic device including at least one delivery optical fiber and at least one collection optical fiber, the delivery optical fiber being configured to deliver light to specimen via a distal end of the fiber optic device, and the collection optical fiber being configured to collect scattered light from the specimen; and an optical beam-shaping element disposed at the distal end of the fiber optic device and configured to focus the light from the delivery optical fiber that enter through a first surface of the optical beam-shaping element and exits through a second surface of the optical beam-shaping element. The system further includes a detector optically or electronically coupled to the probe and configured to receive a signal from the probe, wherein at least one of the first surface and the second surface have an aspheric shape.

Further scope of the applicability of the described methods and systems will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present disclosure may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 shows a schematic of a cell culture vessel having one or more monitoring windows for non-invasive measurement of a cell culture within the vessel, according to embodiments of this disclosure.

FIG. 2 is a cross-sectional view of a monitoring probe and a monitoring window in a wall of a cell culture vessel, according to embodiments of this disclosure.

FIG. 3 is an example graph of Raman spectral measurements of a vessel, monitoring window, and cell culture components, according to embodiments of this disclosure.

FIG. 4 is the example graph of FIG. 3 with spectral zones for measuring glucose and lactic acid, according to embodiments of this disclosure.

FIGS. 5A-5D show cross-section views a peripheral portion of a monitoring window where it is fitted to a cell culture vessel and methods of mitigating warp in the monitoring window, according to embodiments of this disclosure.

FIG. 6 shows a graph of examples of deformation resulting in monitoring windows according to the embodiments shown in FIGS. 5A-5D.

FIG. 7 illustrates a perspective view of an example of a monitoring layer for non-invasive measurement of a cell culture that supports remote monitoring in accordance with embodiments of the present disclosure.

FIG. 8 illustrates an example of a monitoring module in accordance with embodiments of the present disclosure.

FIG. 9 illustrates a perspective view of an example of a stacked cell culture vessel system for non-invasive measurement of a cell culture that supports remote monitoring in accordance with embodiments of the present disclosure.

FIG. 10 illustrates an example of an indentation of a monitoring layer in accordance with embodiments of the present disclosure.

FIG. 11 shows a spherical lens optical probe according to the prior art.

FIG. 12 is a schematic illustration of a spherical lens as shown in the probe of FIG. 11.

FIG. 13 is a schematic illustration of an aspheric optical element according to embodiments of this disclosure.

### DETAILED DESCRIPTION

The singular forms “a,” “an” and “the” include plural referents unless the context clearly dictates otherwise. The endpoints of all ranges reciting the same characteristic are independently combinable and inclusive of the recited endpoint. All references are incorporated herein by reference.

As used herein, “have,” “having,” “include,” “including,” “comprise,” “comprising” or the like are used in their open-ended sense, and generally mean “including, but not limited to.”

All scientific and technical terms used herein have meanings commonly used in the art unless otherwise specified. The definitions provided herein are to facilitate understanding of certain terms used frequently herein and are not meant to limit the scope of the present disclosure.

The present disclosure is described below, at first generally, then in detail on the basis of several exemplary embodiments. The features shown in combination with one another in the individual exemplary embodiments do not all have to be realized. In particular, individual features may also be omitted or combined in some other way with other features shown of the same exemplary embodiment or else of other exemplary embodiments.

Cell culture systems that allow for certain measurements to be completed in real time without disturbing the cells, or in other words, closed systems, may facilitate maintaining a sterile cell growth environment. For example, a monitoring system that is external to a cell culture chamber may provide a non-invasive method for measuring cell status, such as cell growth and health, without directly contacting the cells and without contaminating the growth environment. As used herein, the term “closed system” refers to a system wherein the contents of the system are not open to the surrounding atmosphere. The system may include a closure apparatus, such as a cap, which limits or prevents the introduction of contaminants from the surrounding atmosphere. The system may be, but is not necessarily, sealed to ensure sterility of the contents of the system.

Embodiments of this disclosure include cell culture vessels and systems that allow for monitoring of closed systems via a window provided in the vessel wall. In some embodiments, the cell culture system provides a vessel with a defined window through which the monitoring system analyses the interior of the cell culture chamber. For example, an aspect of one or more embodiments includes a polymer window in the system that allows an external monitoring technology to monitor aspects in the interior of the closed system containing liquid media and biological components or cells. Certain analytical methods, such as infrared (IR) and Raman spectroscopy, are known in the art to be capable of measuring certain factors relevant to the cell culture, such as measuring glucose and lactate in the cell culture. However, these methods generally require the insertion of a probe into the cell culture media, either in situ or by pushing the probe through a port designed for it. Taking spectroscopic readings through a polymer window has not been shown to be practical due to the background noise generated by polymers. For example, the carbon-carbon bonds in a polymer may introduce background signals that can obscure the sensitivity of the signals coming from the glucose and lactate. Nonetheless, embodiments of this disclosure are directed to the use of a polymer window for monitoring (e.g., Raman spectroscopy) through which media components, such as glucose, and cell culture waste products, such as lactate, can be monitored and/or read optically, as well as vessels that contain such windows and the processes and methods used to measure through these windows. As used herein, “monitoring probe” or “monitoring module” includes any device used to measure an aspect of a media layer within a vessel, and may include, for example, an analyte monitor or probe, a spectral probe, including a Raman spectroscopy probe, or other monitors or probes used by those skilled in the art of cell culture. As used herein, “media layer” means a portion of media, whether in solid, liquid, or gaseous form, that is within a vessel and that is the desired target for monitoring. Media layer includes a cell culture or a cell culture media, including components thereof, within the vessel.

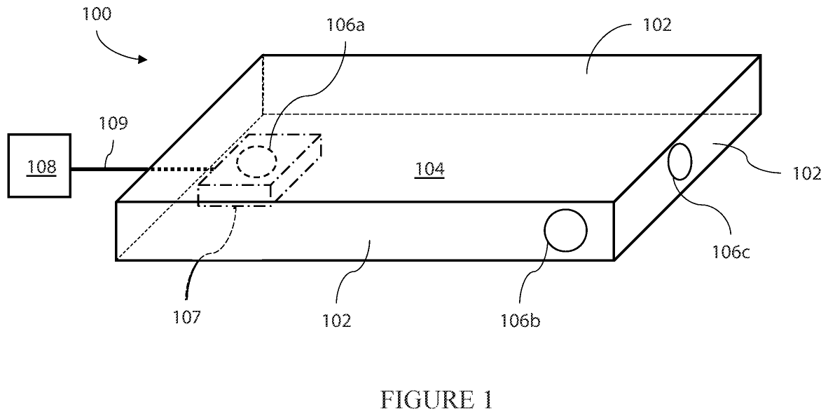

FIG. 1 shows an example of a cell culture vessel 100 formed by one or more side walls 102 that enclose a cell culture space 104 in the interior of the vessel 100. The cell culture vessel is provided with a window 106a in one side wall 102. The vessel 100 is shown with a number of windows 106a, 106b, and 106c to illustrate that the window can be placed as desired on the vessel, and optimal locations may be selected based on the specifics of a given cell culture system. In some cases, multiple windows can be provided on a given vessel so that the location of monitoring at any given time can be chosen at will, a single probe can be used to monitoring the cell culture space 104 via the various windows at different times, or multiple monitoring probes can simultaneously monitoring the cell culture space via the various windows. A monitoring probe 107 is positioned adjacent to the window 106a to measure one or more aspects of a media layer within the cell culture space 104 through the window 106a. The detection system 108 may include a detector and/or processing unit to analyze measurements taken by the probe 107. The probe 107 is connected to a detection system 108 via a signal carrier 109. The signal carrier 109 can be any means of carrying a signal from the probe 107 to the detection system 108, including, for example, an electrode, optical fiber, or wireless signal. According to embodiments, the probe 107 can be a Raman spectroscopy probe.

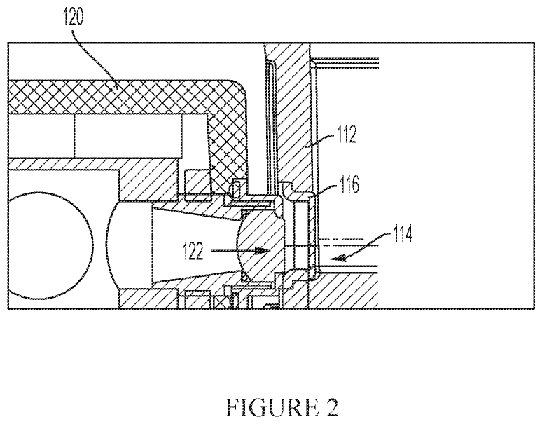

FIG. 2 shows a close-up view of the interface between a monitoring probe—in the example of FIG. 2, a Raman probe—and a window of an example vessel (e.g., a Corning CellStack®), according to one or more embodiments of this disclosure. In FIG. 2, a wall 112 of a cell culture vessel is provided with an opening 114 in which the window 116 is provided. A Raman probe 120 is positioned so that a lens 122 of the probe is adjacent to the window 116 for spectroscopic monitoring of the interior of the cell culture vessel.

As an aspect of one or more embodiments, the window may include one or more polymers having a composition that reduces background signals generated by the polymer during spectroscopy. In particular, background signals are reduced in spectral areas corresponding to the components within the closed system that are the desired targets of the monitoring. For example, the polymer window may be formed from one or more polymers that contain little or no carbon-carbon covalent bonds to avoid generation of background signals in this region of the vessel wall that can reduce measurement sensitivity of organic molecules, which also contain carbon-carbon covalent bonds, that are dissolved in the media layer being monitored. The lack of carbon-carbon bonds reduces or eliminates background signals generated by the polymer during spectroscopy in regions where a detection signal may be generated by organic media components (such as glucose and lactate, for example) that are of interest in the monitoring of the closed system. An example of such polymer material is silicone or polydimethylsiloxane (PDMS). As discussed herein, PDMS is shown to be suitable as a window when measuring glucose and lactate, according to some embodiments. However, embodiments are not limited to this material, and any polymer with reduced or no carbon-carbon covalent bonds may be suitable. Thus, where reference is made to PDMS specifically, it is intended that such other suitable polymers can be substituted for PDMS in some embodiments.

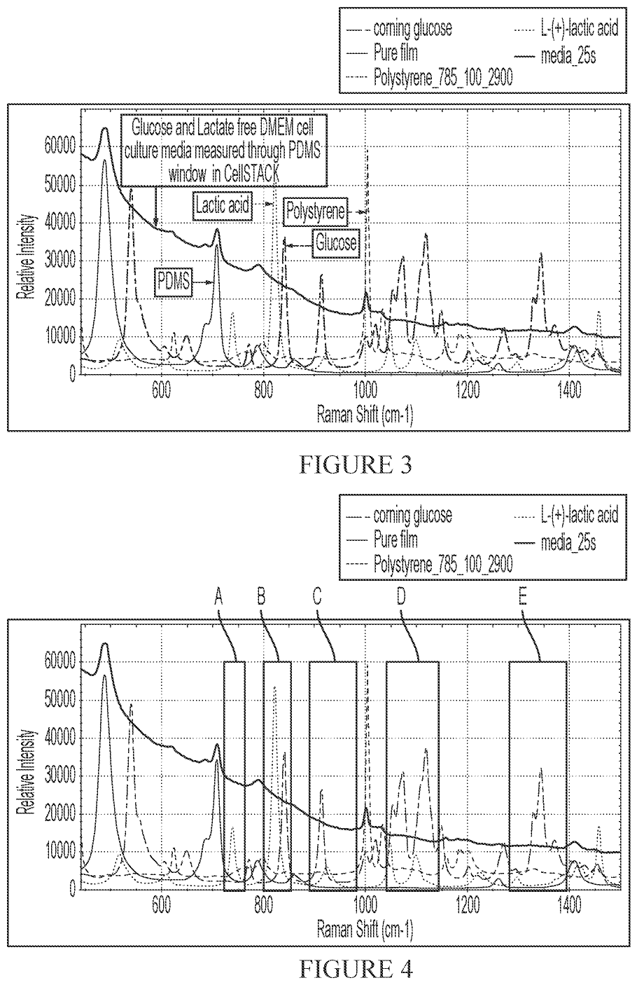

FIG. 3 shows Raman spectral overlay measurements in the CellSTACK® example depicted in FIG. 2. The spectra shown include the background from the silicone window (PDMS), the vessel's polystyrene walls, and the DMEM media (i.e., without glucose and lactate). Also depicted are the signals generated from solutions of glucose and of lactic acid. Comparing the glucose and lactic acid spectra with the DMEM measured through the window in the vessel wall demonstrates that there are peak-free zones in the DMEM spectra which allow for background-free glucose and lactic acid quantification. For example, FIG. 4 shows spectral zones A, B, C, D, and E that allow for quantification of glucose and lactic acid when measured with Raman spectroscopy through a cell culture vessel wall due to the use of a sealed, integrated silicone window, according to embodiments of this disclosure. In other words, the spectra of glucose and lactic acid are discernable above the background spectra of the polymer window, such that the window of embodiments of this disclosure can be used without impairing the monitoring of glucose and lactic acid within the vessel.

The measurements shown in FIGS. 3 and 4 were conducted using a Raman spectroscope and a vessel with an integrated silicone window (i.e., PDMS). The PDMS used to fabricate the window was Dow C6-150 and window thicknesses from 0.005 to 0.020 inches were evaluated, all of which demonstrated good ability to function as a Raman window in the vessel. In some examples, thinner windows may perform slightly better in terms of reducing the amount of material between the spectroscope and the media layer within the vessel. Measurements of glucose and lactic acid were made through the vessel wall window as these are of specific interest to cell culturists. However, embodiments are not limited to the detection of glucose and lactic acid, as other components may be measured using the window and cell culture systems according to embodiments of this disclosure.

The entire cell culture vessel in some embodiments can be made from the same material as the window (e.g., silicone or PDMS). However, it may be advantageous for a vessel to be made from a different material than the window due to physical and chemical characteristics or requirements of the vessel material. For example, polystyrene is sometimes used, for example, in roller bottles, tubes, petri-dishes, flasks, CellSTACKS and HYPERStacks; polycarbonate in shaker flasks; polyester in shaker flasks and media bottles; and polypropylene in tubes and cryovials. When it is desired to use a polymer for the vessel that differs from the polymer of the window, the polymer window can be assembled into the wall using assembly sealing methods known in the art, including, for example, with the use of adhesives, thermal sealing with compatible polymers, or via compression sealing, where the elastomeric properties of the PDMS allow for the generation of the compression seal.

Because silicone is an elastomer, embodiments of this disclosure provide the additional advantage of the window acting as a gasket within the provided opening of the vessel to seal the opening an maintain the integrity of the closed system. This is especially advantageous in cases where the vessel is made from a polymer or other material that is not compatible with certain types of sealing methods, such as creating a thermal seal between the vessel and polymer window materials. According to one or more embodiments, the polymer or silicone window can be joined to the vessel wall using a compression fit of the silicone within the opening, lamination, adhesives, or other methods known in the art. By having a sealed window through which the targeted components can be effectively monitored, the closed system remains closed to contamination or external probes; moving or opening the vessel is not necessary; frozen solutions can be monitoring (e.g., for cell viability) without thawing; minimum or no calibration of probes is necessary; and monitoring can be performed in real time, avoiding the need to remove the vessel from incubators, for example, and avoiding the need to alter the system being analyzed by removing solution. In addition, silicone windows can be inexpensively manufactured, and can be sterilized by standard processes in the industry, such as gamma or e-beam radiation. Thus, such windows are practical and suitable for all cell culture vessels, including disposable vessels. In addition, in a vessel using an elastomeric polymer window, the distal optical component of the monitoring unit's probe can be pressed up against the window itself to minimize the distance between the optic and object being analyzed in the interior of the cell culture chamber.

In some cases, the fitting of the polymer window within the opening of the vessel can cause the polymer window to warp. For example, the polymer window may be compressed to fit within the opening and/or may be compressed by sealing ring or retaining mechanism placed on a peripheral part of the window. Such compression of the polymer induces stress within the polymer window, which can cause the window to warp or bend. This raises the risk of the window not having a flat surface through which monitoring is performed. If the window is warped in this way, it is possible that monitoring results could be affected and/or precise placement of the probe near or against the window could be impaired. Accordingly, embodiments of this disclosure provide aspects to minimize the bowing or warping of the window without comprising the seal between the polymer window and the cell culture vessel.

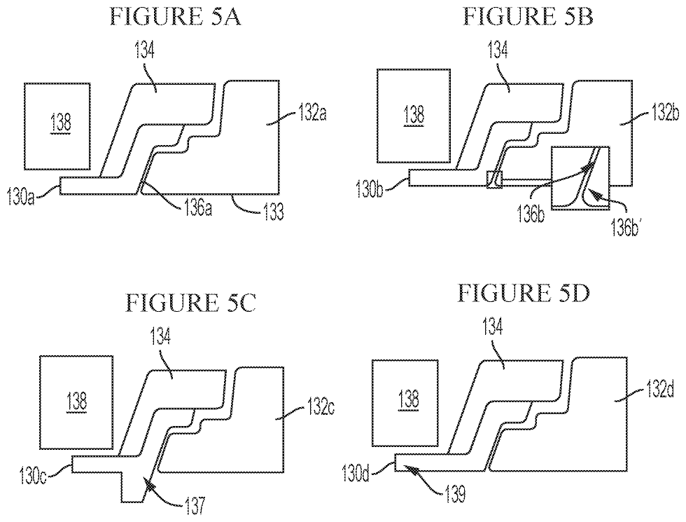

FIGS. 5A, 5B, 5C, and 5D show close-up cross-section views of peripheral portions of windows 130a, 130b, 130c, and 130d where they are joined to cell culture vessels 132a, 132b, 132c, and 132d, respectively, according to one or more embodiments. A monitoring probe 138 is shown on an exterior side of the windows and adjacent to the viewing portion of the windows 130a-103d, while a peripheral portion of each window 130a-130d is shown in a position where it is to be compressed against an edge of the cell culture vessels 132a-132d by a compression ring 134. Applicant has found that, due to the compression of the peripheral portion of a window between the compression ring 134 and the cell culture vessel 132a-132d, the viewing portion of the window facing to the monitoring probe 138 can warp or bow. Applicant has also found that the stresses in the window can be controlled by controlling the interfacing of the peripheral portion of the window with the compression ring 134 and the edge of the cell culture vessel. For example, in FIG. 5A, the viewing portion of the window 130a is flat before the peripheral portion is compressed between the compression ring 134 and the edge of the vessel 133, but depending on the angle of the taper 136a at the interior edge of the vessel, the viewing portion of the window 130a may warp so that it is no longer flat after the window 130a is compressed between the compression ring 134 and the vessel 132a. In one example, a single taper 136a of 21° at the interior edge of the vessel opening was found to induce curvature in the window, where the angle of taper is the angle between the edge of the vessel and a line that is vertical with respect to FIG. 5A-5D (i.e., a line perpendicular to the inner surface 133 of the vessel and in the plane of the page showing FIGS. 5A-5D). However, according to aspects of embodiments, this curvature can be prevented or corrected. FIGS. 5B, 5C, and 5D show examples of embodiments that control the shape of the window to mitigate the induced curvature.

In FIG. 5B, for example, the edge of the vessel 132b is provided with a compound taper. As used herein, “compound taper” means a taper with a non-constant angle, including two or more distinct taper angles or a variable-angle taper. In FIG. 5B, the compound taper includes an outside taper 136b at a first angle corresponding to the taper 136a of FIG. 5A, and an inside taper 136b′ at a second angle different from the first angle. In some embodiments, the inside taper 136b′ is smaller than the outside taper 136b. In one embodiment, the inside angle 136b′ is 10° and the outside taper 136b is 21°. These angles are given for example only, and it is understood that the compound taper can be adjusted based on the specific window and vessel arrangement to achieve a desired result. According to one or more embodiments, the taper of the outside angle 136b or the inside angle 136b′ is less than 90° and greater than 0°, is less than or equal to about 45° and greater than or equal to about 2°, is less than or equal to about 30° and greater than or equal to about 5°, is less than or equal to about 25° and greater than or equal to about 10°, is less than or equal to about 21° and is greater than or equal to about 10°.

In FIG. 5C, warping of the window 130c is reduced by increasing the stiffness of the window 130c at least in the portion beneath the compression ring 134. The increased stiffness may be accomplished by altering the composition of the window in the peripheral portion to increase the material stiffness. Alternatively, or additionally, the stiffness may be increased by altering the physical shape of the peripheral portion. For example, as shown in FIG. 5C, the peripheral portion of the window 130c has a portion 137 of increased thickness relative to the viewing portion of the window 130c. The portion 137 of increased thickness alters the stiffness of that section and the stresses resulting from compressing the peripheral portion of the window 130c. As a result, the viewing portion of the window 130c can maintain good flatness even after being sealed between the compression ring 134 and the vessel 132c. In some preferred embodiments, the stiffness is created only under the peripheral portion to avoid the stiffness change resulting in an altered monitoring sensitivity in the center portion of the window.

FIG. 5D shows an example of another solution to warping of the window. Specifically, the window 130d can be formed with a pre-existing negative bow 139 or curvature. That is, before the window 130d is compressed between the compression ring 134 and the vessel 132d, the viewing portion of the window 130d is not flat, but instead bows slightly. This negative bow 139 will counter the tendency of the window 130d to bow when compressed by the compression ring so that the final product will have a window having a desired degree of flatness. In other words, the negative bow 139 becomes flatter as the peripheral portion of the window is compressed by the compression ring. In some embodiments, the negative bow 139 can make the viewing portion of the window bow towards the interior of the vessel 132d. In other embodiments, the negative bow can make the viewing portion of the window bow towards the exterior of the vessel, in a case where the induced warp from the compression ring 134 causes the window to bow towards the interior of the vessel. In some preferred embodiments, the degree of bowing created in the window can correspond to the degree of warping induced by the compression ring. For instance, in an example of the configuration of FIG. 5A, the window deformed and bowed upward by about 50 μm. Thus, a negative bow of about 50 μm to counteract the same degree of deformation was shown to produce a final window of the desired flatness. This amount of bowing is given as an example only, and it is understood that the amount of bowing can be adjusted to adequately counteract the induced warp in the final window. In some examples, the negative bow or curvature may be from about 10 μm to about 1000 μm, up to about 500 μm, up to about 400 μm, up to about 300 μm, up to about 200 μm, up to about 100 μm, or from about 10 μm to about 50 μm.

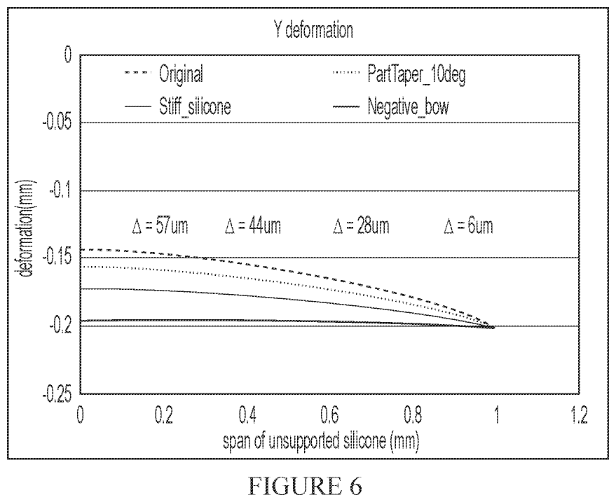

FIG. 6 shows a graph plotting examples of the warp in monitoring windows according to the embodiments shown in FIGS. 5A-5D. The “original” configuration corresponds to the embodiment shown in FIG. 5A and has the highest deformation (about 50 μm). The compound taper (corresponding to FIG. 5B) has less deformation (about 44 μm) than the original configuration; the window with a stiffened region has still less deformation (about 28 μm); and the window with a negative bow has the least deformation (about 6 μm or less) and is almost flat. Thus, it can be seen that these features offer a window with improved flatness.

According to one or more embodiments, the monitoring window is incorporated into the vessel with one or more retaining features disposed near the window or on the vessel, where the one or more retaining features allow for a probe to be “clipped” or removably attached to the vessel in a position where the probe can monitor the closed system via the window. According to another aspect of some embodiments, an alignment feature can be provided that aligns the probe in a location suitable for accurate monitoring of the closed system. The retaining feature and alignment feature can be provided in combination or separately, and, in some embodiments, the retaining feature itself is an alignment feature. In some embodiments, the probe may also be handheld, and the alignment feature can be used to align the handheld probe.

As used herein, “vessel” includes any cell culture vessel, whether used in static or dynamic (e.g., perfused) conditions, and whether used for adherent cells or suspension cells. For example, suitable vessels include T-flasks, multi-layer flasks, CellSTACKS®, Cell Factories®, HYPERFlasks®, shaker flasks, spinner flasks, bioprocess bags, and bioreactors, as well as in auxiliary vessels such as cryotubes, and media bags and bottles used to confirm formulations and quality. The window can also be placed in a tubing path, such as may be used with perfusion vessels, and may be formed either in the tubing itself or integrated into a fitting that is connected or over-molded into the tubing. If integrated into the tubing itself, the window may be formed from a thin-walled section of the tubing (e.g., silicone tubing). For vessels with control systems that allow for dynamic feeding, the real-time measurements can be advantageous by enabling fine-tuned control of the feeding system.

The cell culture vessels described herein may be adherent cell culture vessels generally including a planar surface on which cells adhere while being cultivated. In some embodiments, the cell culture vessels may be suspension cell culture vessels, in which cells are cultured in suspension. In some embodiments, the cell culture vessels may be configured for culturing cells adhered to surfaces or carriers that are in suspension. Such carriers may include, for example, microcarriers that may be dissolvable or digestible to release the cells adhered thereto. A stacked cell culture vessel may be used with multiple layers for cell culture, providing an increased area for cell growth over single layer vessels or dishes. In a stacked cell culture vessel, one or more layers may be a monitoring layer in which cells can be monitored by a monitoring module external to the closed system. The monitoring layer may be positioned at the top or bottom of the stacked vessel, and may also be between other cell culture layers within a stacked cell culture vessel, and take measurements of cell culture chambers of the various layers of the stack.

As described herein, a cell culture vessel may include a monitoring layer configured to allow spectral analytical technology and/or optical technology (e.g., micro lens arrays and waveguides) to monitor cells or media that are within the monitoring layer and are part of a closed system. Embodiments of the present disclosure further include a monitoring module including at least one of spectral analytical technology and optical technology. As will be described in more detail below, the monitoring module may include spectral analytical technology and/or optical technology integrated into the monitoring module. Embodiments of the present disclosure allow for the monitoring of cell confluence and measuring analytes with spectral interrogation that illuminates, receives, and processes signature wavelengths through a window in the vessel that maintains the closed nature of the cell culture system and that allows the spectra of the monitoring targets to be discernable above the background spectra of the window itself.

According to some embodiments, a cell culture vessel may include a monitoring layer including at least one indentation, the at least one indentation being configured to receive at least one of monitoring module. The monitoring module can include at least one of an optical technology (e.g., micro lens arrays and waveguides) and a spectral analytical technology. Spectral analytical technology, as used herein, includes Raman spectroscopy and the monitoring module can thus include a Raman spectroscopic probe.

Embodiments of the present disclosure provide for closed-system operation of a cell culture vessel with a monitoring module disposed external to a cell culture chamber. Embodiments of this disclosure provide for monitoring of a closed-system cell culture vessel via a monitoring window provided in the vessel. The monitoring window can improve monitoring compared to conventional vessels.

Embodiments of the present disclosure allow for the transmission of cell status from the monitoring layer to a user in a remote location. An aspect of some embodiments includes a communication component to transmit monitoring data from the monitoring module to a user in a remote location. This configuration may be implemented in single use or multi-use stacked vessels. The closed system remains sterile and able to continuously grow cells, for example by remaining in an incubator, while taking real-time cell status data.

The monitoring layer as described herein may be made of polystyrene. In conjunction with the monitoring module, the monitoring layer may allow for two monitoring functions of the cell growth areas: cell confluence and analyte measurement. The confluence monitor may employ a dual lens system with a mirror formed within the monitoring module, and an attached camera may provide light, image capture, magnification, and image delivery to a user. The analyte monitor may include a spectral analytical technology system and may further include a waveguide system with diffraction grating and lens in the monitoring module. Fibers for excitation and emission may be attached to the monitoring module and may also be connected to a spectral sensor system. Embodiments may include either one or both of cell confluence and analyte measurement monitoring.

An exemplary confluence monitor may employ a dual lens system with a mirror for reflecting light to a cell growth surface with a cell culture chamber for illumination and image capture. The camera may provide the light and image capture function. Light waves or beams may travel through the lens to the mirror where it is focused on an area within the cell growth area. The illuminated image is then received by the camera once passing through the lens.

An exemplary analyte monitor may include a waveguide array. The monitor may employ dual optical ports where one port may be for excitation light and the other port may be for emission light. The excitation light may travel along the light guide (e.g., waveguide) to the diffraction grating and lens where it reflects off the diffraction grating into the media of a cell culture chamber. The emission fiber may receive the light from the excitation state of the media and deliver the excitation light to the spectral sensor (e.g., detector) to produce an emission or adsorption spectrum. The spectral sensor may include a 2D detector array system.

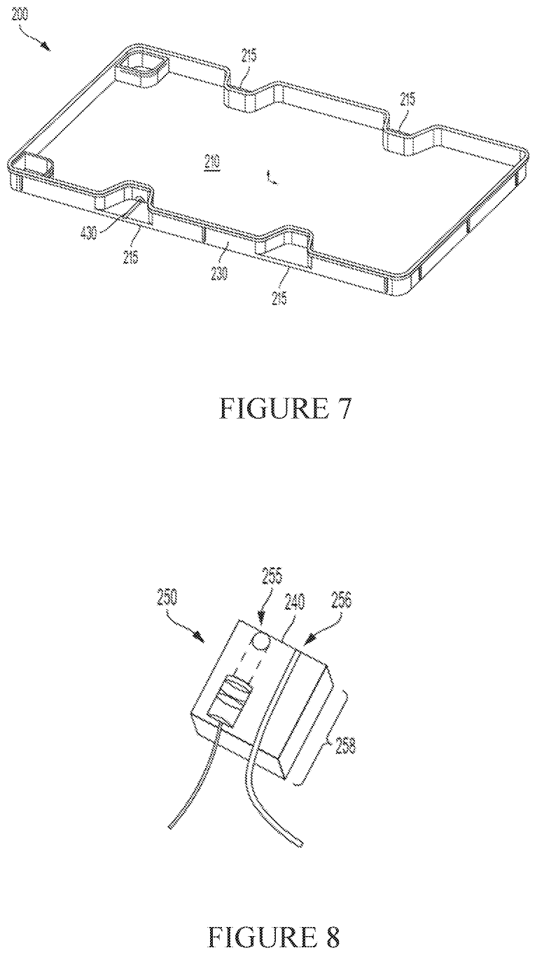

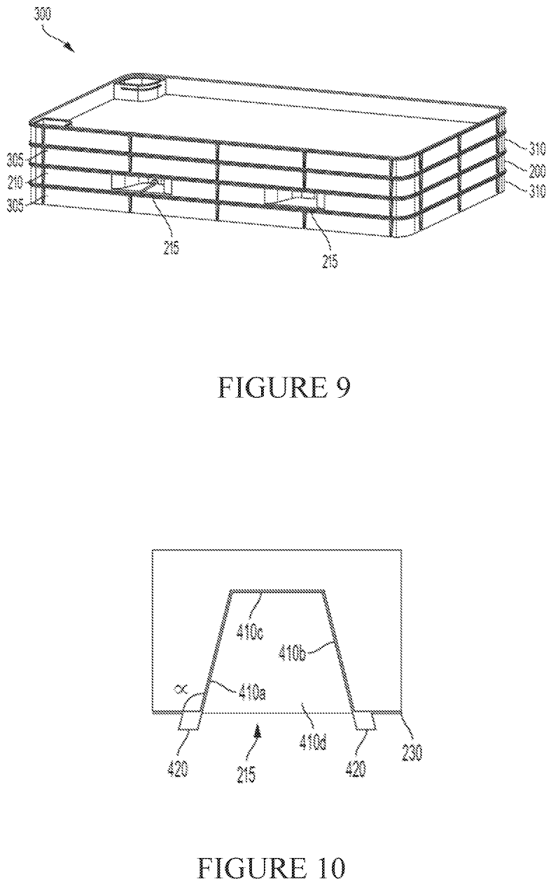

FIG. 7 shows a perspective view of a monitoring layer for non-invasive measurement of cell culture chambers that supports monitoring according to one or more embodiments of the present disclosure. The monitoring layer 200 includes an outer wall 230 surrounding a cell culture chamber 210, and at least one indentation 215 extending inward from the outer wall 230 toward the interior of the cell culture chamber 210. While the monitoring layer 200 shown in FIG. 7 includes four indentations 215, it should be appreciated that monitoring layers 200 in accordance with embodiments of the present disclosure may include any number of indentations 215. As will be explained in more detail below, the indentations 215 are configured to receive a monitoring module 250 (e.g., probe 107 in FIG. 4). As such, the indentations 215 and the monitoring module 250 may have corresponding shapes. As will further be explained in more detail below, the monitoring layer 200 may also include retaining features which cooperate with the monitoring module 250 to maintain the monitoring module 250 in the indentations 215. Optionally, the monitoring layer 200 may include one or more alignment features 430, and the alignment feature 430 can be distinct from or integrated with a retaining feature. The monitoring layer 200 may be configured to operate in a wide temperature range, for example the monitoring layer 200 may operate in an incubator configured for cell growth. In some examples, the monitoring layer 200 may be part of a stacked cell culture vessel as shown in FIG. 9.

FIG. 8 illustrates a monitoring module in accordance with embodiments of the present disclosure. As described herein, the monitoring module 250 may include a head portion 258 having a front face 240 which is configured to contact an inner wall 410c of an indentation 215 of the monitoring layer 200. The monitoring module 250 further includes at least one of a confluence monitor 255 and an analyte monitor 256. It should be appreciated that the monitoring module 250 may include one of a confluence monitor 255 and an analyte monitor 256 or, alternatively, as shown in FIG. 8, may include both of a confluence monitor 255 and an analyte monitor 256. The confluence monitor 255 may be configured to measure cell status in the cell culture chamber 210 of the monitoring layer 200, or may be configured to measure cell status in a cell culture chamber 305 of a cell culture layer 310 positioned above or below the monitoring layer 200 (see FIG. 9). Similarly, the analyte monitor 256 may be configured to monitor analytes in the cell culture chamber 210 of the monitoring layer 200, or may be configured to monitor analytes in a cell culture chamber 305 of a cell culture layer 310 positioned above or below the monitoring layer 200. Where the monitoring module 250 includes both a confluence monitor 255 and an analyte monitor 256, both monitors 255, 256 may be configured to monitor the cell culture chamber 210 of the monitoring layer 200, or both monitors 255, 256 may be configured to monitor at least one cell culture chamber 305 of a cell culture layer 310 positioned above or below the monitoring layer 200. Optionally, where the monitoring module 250 includes both a confluence monitor 255 and an analyte monitor 256, one of the confluence monitor 255 and the analyte monitor 256 may be configured to monitor the cell culture chamber 210 of the monitoring layer 200 and the other of the confluence monitor 255 and the analyte monitor 256 may be configured to monitor at least one cell culture chamber 305 of a cell culture layer 310 positioned above or below the monitoring layer 200.

According to embodiments of the present disclosure, the monitoring module 250 may have a shape corresponding to the shape of the indentation 215 of the monitoring layer 200 such that the monitoring module 250 may be received into the indentation 215. The indentation 215 and monitoring module 250 are designed so that an analyte monitor and/or confluence monitor of the monitoring module 250 may be positioned to face a polymer window provided in the vessel, so that a media layer within the vessel may be measured through the window. As shown in FIG. 10, the indentation 215 may have sidewalls 410a and 410b and an inner wall 410c. The sidewalls 410a, 410b may extend from the outer wall 230 of the monitoring layer 200 to the inner wall 410c of the indentation 215 at an angle a that is greater than about 90 degrees such that the indentation 215 has an isosceles trapezoid shape. The head portion 258 of the monitoring module 250 may have a corresponding isosceles trapezoid shape, or may have an orthogonal shape where the front face 240 of the head portion 258 has a width that is no greater than the width of the inner wall 410c of the indentation 215. Alternatively, the sidewalls 410a, 410b may extend perpendicular to the outer wall 230 of the monitoring layer 200 and parallel to each other. As such, the head portion 258 of the monitoring module 250, such as is illustrated in FIG. 8, may have an orthogonal shape which corresponds to the shape formed by the sidewalls 410a, 410b extending perpendicular to the outer wall 230. Alternatively, the sidewalls 410a, 410b may have a concave shape and the head portion 258 of the monitoring module 250 may have a rounded feature configured to be received in the concave-shaped sidewalls 410a, 410b of the indentation 215. The shapes of the indentations 215 of the monitoring layer 200 and the head portion 258 of the monitoring module 250 discussed above are meant merely as examples. The indentations 215 may have any shape and the monitoring module 250 may have any corresponding shape such that the monitoring module 250 may be received in the indentation 215 and that the front face 240 of the head portion 258 contacts the inner wall 410c of the indentation 215.

The confluence monitor 255 may optically capture the cell status of the cells in the cell culture chamber 104, 210, 305 and the analyte monitor 256 may optically capture analyte status in the cell culture chamber 104, 210, 305. The confluence monitor 255 and the analyte monitor 256 may include a communication component for transmitting data, such as cell status data or analyte status data, from the monitors to a remote location via a wired communication network or a wireless communication network. For example, the communication component of each monitor may include a Wi-Fi transceiver.

FIG. 9 shows a perspective view of a stacked cell culture vessel system 300 that can be used, in conjunction with a monitoring module 250, for non-invasive measurement of a cell culture chamber 104, 210, 305 in accordance with embodiments of the present disclosure. The stacked cell culture vessel system 300 may include a plurality of cell culture layers 310 and at least one monitoring layer 200 with one or more polymer windows as described above.

As illustrated, the stacked cell culture vessel system 300 may include any number of cell culture layers 310 and any number of monitoring layers 200. As shown in FIG. 9, the cell culture vessel system 300 may include a cell culture layer 310 below a monitoring layer 200 and a cell culture layer 310 above the monitoring layer 200. Where the cell culture vessel system 300 includes a plurality of monitoring layers 200, the system 300 may include any number of cell culture layers 310 between any two of the plurality of monitoring layers 200. For example, the cell culture vessel system 300 may include between 1 and 50 cell culture layers 310 between each monitoring layer 200, such as between or 2 and 40 cell culture layers 310, or between 3 and 35 cell culture layers 310, or between 5 and 30 cell culture layers 310, or even between 10 and 25 cell culture layers 310 between each monitoring layer 200, and all values therebetween. It should be appreciated that the number of cell culture layers 310 between different sets of the plurality of monitoring layers 200 may vary within the same stacked cell culture vessel system 300. Additionally, the stacked cell culture vessel system 300 may be configured to operate over a wide temperature range such as in an incubator at a temperature designed for cell growth.

FIG. 10 further illustrates exemplary retaining features in accordance with embodiments of the present disclosure. As shown, the outer wall 230 of the monitoring layer 200 includes clips 420 at the edges of the opening formed by the sidewalls 410a and 410b of the indentation 215. The clips 420 are configured to fit into a corresponding receptor on the monitoring module 250, thus maintaining the monitoring module 250 within the indentation 215. A base portion 410d of the indentation 215 can include a raised channel 430, as shown in FIG. 7. The raised channel 430 is configured to fit into a corresponding notch on the bottom of the monitoring module 250, thus maintaining the monitoring module 250 within the indentation 215. As another option, the retaining feature may be a biased retention clip (not shown) on at least one surface of the monitoring module 250. The biased retention clip may have a similar design and function as those known to be used for telephone line connectors and Ethernet cable connectors. The indentations may include at least one clip groove (not shown) which receives a corresponding biased retention clip on a surface of the monitoring module 250 and which, in conjunction with the biased retention clip, limits motion of the monitoring module 250 and maintains the monitoring module 250 within the indentation 215.

According to embodiments of the present disclosure, the confluence monitors 255 and the analyte monitors 256 may capture the cell status of the cells and analyte status of the media in cell culture chambers 104, 210, 305, including inter-layer measurements and monitoring, through a polymer window provided in the vessel. In some cases, a single confluence monitor 255 may monitor cell status of the cells of multiple stacked cell culture chambers 104, 210, 305, or a single analyte monitor 255 may monitor analyte status of the media of multiple stacked cell culture chambers 104, 210, 305.

According to embodiments of the present disclosure, a confluence monitor 255 may take measurements of the cells in a cell culture chamber 104, 210, 305 by any optical means. For example, the confluence monitor 255 may include a 2D imaging array to monitor cells in the cell culture chamber 104, 210, 305. Alternatively, the confluence monitor 255 may include a multi-lens (e.g., dual lens) system with at least one mirror and at least one camera. An exemplary confluence monitor may include one or more optical paths, lenses, and mirrors that may be configured to use a number of illumination options (e.g., reflected light illumination, epi-illumination, dark field illumination, light field illumination, etc.) to observe the cells. Light beams may be transmitted from a camera through a first lens, where the light beams may be refracted and focused towards a mirror. Once the light beams contact the mirror, the light beams may be reflected at any angle, for example about 90 degrees, to be directed through a second lens into a cell culture chamber 104, 210, 305 to measure the confluence of cells. The camera may capture the illuminated cells to produce an image of their real-time confluence that may be used to monitor cell growth over time. Through such an optical arrangement, a confluence monitor designed to image at least one cell culture chamber 104, 210, 305 above or below the monitoring layer 100, 200. As media in the cell culture chambers 104, 210, 305 may affect image quality, the confluence monitor 255 may take measurements of the cell culture chamber 305 above the monitoring layer 200 in order to image the cells on the side of the cell culture chamber 305 that has less media.

Optionally, the confluence monitor 255 may include a fiber probe (e.g., a dual clad fiber two multi-mode fibers (MMFs), or a multicore fiber) to direct light beams to the cell culture chamber 104, 210, 305 and to transmit cell images to the camera or detector. Additionally, image magnification to monitor cell confluence may be performed external to the monitoring module 250. For example, a light pipe may be used within the confluence monitor 255 to transfer the cell surface image without magnification to an external microscope at a location remote to the monitoring module 250.

According to embodiments of the present disclosure, the confluence monitor 255 may take measurements of the health of the cells by measuring the composition of the media within a cell culture chamber 104, 210, 305 by any spectral means (e.g., Raman spectroscopy). According to some embodiments, the analyte monitor 256 may include a waveguide (e.g., a light pipe) and detector. The waveguide delivers light to the media within the cell culture chamber 104, 210, 305. Optionally, the analyte monitor 256 may include a diffraction grating and lens which may receive excited light from the waveguide and direct the excited light to the media within the cell culture chamber 104, 210, 305. Excited light may be produced in a number of ways. Based on the composition of the media, distinct emission spectrums will be given off and captured by the detector. The detector may transmit the captured emission or adsorption spectrum to a user. The user may use software to determine the composition of the media based on the emission or adsorption spectrums. Some examples of analytes that may be measured by analyte monitor include glucose, lactose, and glutamine.

According to embodiments of the present disclosure, the analyte monitor 256 may include a light emitting diode (LED) or laser. The LED or laser may be paired with a photodiode detector within the analyte monitor 256.

The analyte monitor 256 can be designed to image the cell culture chamber 104, 210 of the monitoring layer 100, 200. However, as discussed above, the analyte monitor 256 may be designed to monitor at least one cell culture chamber 305 above or below the monitoring layer 200. A diffraction grating and lens may be utilized to direct light in the waveguide to at least one cell culture chamber 305 above or below the monitoring layer 200. It is preferable for the analyte monitor 256 to transmit excited light into the media while passing through as few other materials as possible in order to produce a clean emission spectrum. However, as discussed above, embodiments of this disclosure provide a polymer window that allows monitoring to be performed through the window while targeted analytes are still detectable via spectroscopy due to the unique features of the polymer window.

Embodiments of this disclosure relate to optical devices, systems, and methods for monitoring cell cultures using spectral analytical technology (e.g., Raman spectroscopy). Raman spectroscopy is a useful method of monitoring aspects of a cell culture. In general, a Raman spectroscopy system includes a probe coupled to a plurality of optical fibers. These fibers include at least one excitation fiber that optically couples the probe, possibly via one or more filters, switches, or other optical components, to a radiation source, which may include a laser having an output wavelength from about 200 nm to about 1550 nm. The fibers also include at least emission fiber optically that optically couples the probe, possibly via one or more filters, switches, or other optical components, to a detector. The probe thus delivers radiation from the radiation source, via the excitation fiber, to a sample to be analyzed, and radiation scattered by the sample is collected by the probe and returned, via at least one emission fiber, to the detector.

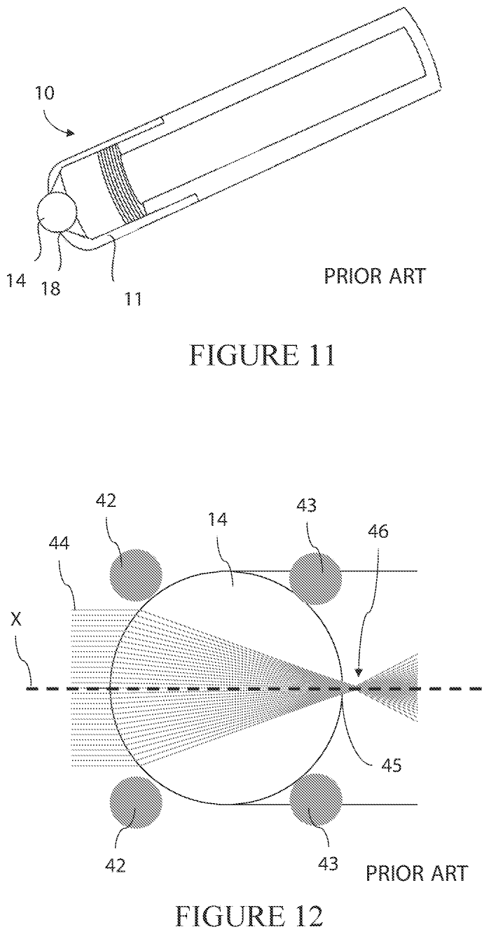

By way of example, FIG. 11 shows a conventional Raman spectroscopy probe 10. The probe 10 includes spherical lens 14 seated within cylindrical probe tip 11 at lens opening 18. A seal between the probe tip and the lens can be formed at the opening by welding or braising and the use of epoxies or other adhesives. Elements such as gaskets, O-rings, and other sealing means may be present to provide a leak-proof system, and may be provided. As shown in FIG. 12, O-rings 42, 43 may be used to seal the spherical lens 14. With respect to FIG. 11, O-ring 43 is placed inside probe tip 11 such that it is seated around lens opening 18 at the distal end of probe tip 11. Lens 14 is also placed inside probe tip 11 such that it is seated on top of O-ring 43 and a portion of the lens extends through lens opening 18 and is external to probe tip 11. Thus, the lens 14 is held in place, and a seal between the lens 14 and the probe tip 11 is formed. In addition, O-ring 42 is seated in probe tip 11 on top of lens 14.

The spherical lens 14 of FIGS. 11 and 12 would be placed on or immersed into the specimen tested, which could be a liquid or powder. As described above, light 44 is transmitted through a proximal surface of the spherical lens, such as that in FIGS. 11 and 12, and the spherical lens 14 will collect light very close to the distal surface 45 of the sphere, as shown by the focus point 46 in FIG. 12. The distance of the focus point 46 from the distal surface 45 will depend on the diameter of the lens or the effective pupil diameter. A wide diameter or pupil diameter will allow more light to enter the spherical lens at the proximal surface and increase the distance from the distal end of the lens 14 that light can be collected (or the distance from the probe that the analysis can be conducted). The diameter of the spherical lens may be, for example, 3 mm. However, the presence of O-rings 42 and 43 reduces the available surface area of the spherical lens 14 that can be used for light transmittance. Thus, the realities of providing a spherical lens that is sealed with an O-ring or other means further limits that distance at which the lens focuses. This limitation prevents use a spherical lens on the end of a probe to view through the wall of a vessel. When it is required for the probe to be inserted into the media being analyzed, the system has a higher risk of contamination or of being physically disturbed.

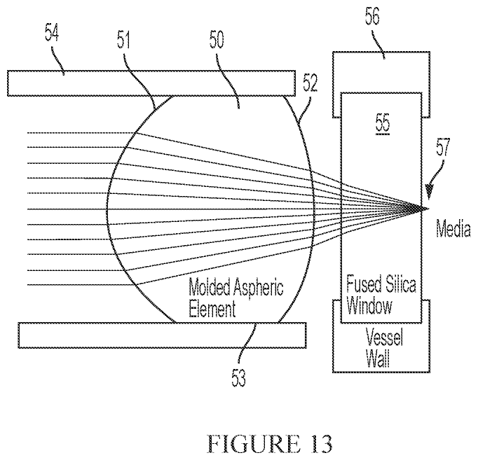

According to embodiments of this disclosure, a custom optical component is provided that allows a Raman spectrometer probe to collect light from a great enough distance that the Raman probe can be used to analyze a cell culture through a vessel wall or window. In contrast to the sphere-shaped lens of FIGS. 11 and 12, the optical component has one or more aspheric surfaces. The aspheric surface minimizes the collection angle loss relative to a spherical lens. As shown in FIG. 13, an optical component 50 is provided within a probe tube 54 and includes a proximal surface 51 with an aspheric shape and a distal surface 52. Due to the optics provided by the aspheric surface, the probe can be used to analyze components at a focus point 57 through a window 55 provided in the wall 56 of a vessel. By analyzing components through a window, the components and/or their environment (e.g., a cell culture and surrounding media) will not be contaminated or disturbed by the analysis.

In contrast to a spherical lens, aspheric lenses or aspheric surfaces according to embodiments of this disclosure are elements with surface profiles that are not portions of a sphere or cylinder, or whose surface geometry deviates from a sphere. An aspheric surface can be a surface having a radius of curvature that varies radially from the center of the lens, or that changes with distance from the optical axis, unlike a sphere, which has a constant radius.

In FIG. 13, the proximal surface 51 is an aspheric surface and the distal surface 52 is spherical. However, embodiments are not limited to this arrangement. In some embodiments, both the distal surface 52 is aspheric and the proximal surface 51 are not aspheric. In other embodiments, both the proximal surface 51 and the distal surface 52 are aspheric. Suitable materials for the optical component 50 include materials that have no or minimal scattering and have little or no luminescence. Suitable materials include silica (e.g., fused silica) and sapphire. According to some embodiments, the optical component includes K-FIR98UV or K-FIR100UV from Sumita Optical Glass.

According to one or more embodiments, the optical component 50 can be sealed within or affixed to a probe tube using any conventional means known in the art, including, for example, welding or braising, epoxies or other adhesives, or elements such as gaskets, O-rings, and other sealing means. According to some embodiments, the optical component 50 may be molded to fit into the probe tube 54.

According to embodiments disclosed herein, the aspheric optical component 50 is provided on the distal end of a probe, which is then held or positioned up to a vessel for analyzing a system within the vessel through a wall or window of the vessel. According to one or more embodiments, the vessel has one or more retaining features disposed near the window or on the vessel, where the one or more retaining features allow for a probe to be “clipped” or removably attached to the vessel in a position where the probe can monitor the closed system via the window or vessel wall. Thus, the optical component can be positioned at a suitable distance from the interior of the vessel to allow analysis of the closed system. According to another aspect of some embodiments, an alignment feature can be provided that aligns the probe in a location suitable for accurate monitoring of the closed system. The retaining feature and alignment feature can be provided in combination or separately, and, in some embodiments, the retaining feature itself is an alignment feature. In some embodiments, the probe may also be hand-held, and the alignment feature can be used to align the hand-held probe.

In an alternative embodiment, the aspheric optical component is provided on the vessel itself. For example, the aspheric optical component can be molded into the wall of the vessel, or affixed adjacent to a window provided in the wall of the vessel. In this way, the closed system of the vessel can remain closed to not risk contamination.

One or more embodiments of this disclosure provide cell culture systems that allow for culturing and monitoring of cells in a closed system. An aspect of some embodiments includes a cell culture vessel having a cell culture chamber confined by one or more walls of the vessel, and a monitoring system disposed on the exterior of the cell culture chamber to analyze the cell culture or the cell culture media. The monitoring system can include a spectral analysis system (e.g., Raman spectroscopy). The vessel wall can be transparent or at least transparent to the spectrum of light or radiation emitted by the monitoring system.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

While the present disclosure includes a limited number of embodiments, those skilled in the art, having benefit of this disclosure, will appreciate that other embodiments can be devised which do not depart from the scope of the present disclosure.

1. A cell culture vessel configured for non-invasive measuring of a cell culture, the vessel comprising:
a cell culture chamber configured to operate as a closed system, the cell culture chamber comprising an interior space configured for housing at least one of the cell culture and a cell culture media;
a wall defining a boundary of the cell culture chamber and separating the interior space of the cell culture chamber from an exterior of the cell culture chamber; and
a window disposed in the wall and separating the interior space of the cell culture chamber from an exterior of the cell culture chamber, the window comprising a polymer and being configured to allow monitoring of the cell culture via a monitoring module disposed on the exterior without the monitoring module coming into physical contact with the cell culture or the cell culture media.

2. The vessel of claim 1, further comprising a monitoring module disposed on the exterior side of the window and configured to monitor an aspect of the cell culture through the window.

3. The vessel of claim 2, wherein the monitoring module comprises an analyte monitor.

4. The vessel of claim 3, wherein the analyte monitor comprises a spectral element configured to emit one or more excitation wavelengths of light and capture emitted light from a media layer within the cell culture chamber.

5. The vessel of claim 4, wherein the spectral element is configured to perform Raman spectroscopy on the media layer.

6. The vessel of claim 3, wherein the analyte monitor comprises a waveguide and a detector.

7. The vessel of claim 6, wherein the analyte monitor further comprises a diffraction grating and lens.

8. The vessel of claim 6, wherein the analyte monitor comprises a fiber probe and a detector.

9. The vessel of claim 3, wherein the analyte monitor is configured to monitor at least one of glucose, lactose, and glutamine within the cell culture chamber.

10. The vessel of claim 1, wherein the window comprises a material with an emission spectra that does not interfere with the monitoring module's detection of analytes in the cell culture vessel.

11. The vessel of claim 1, wherein the window comprises silicone, polydimethylsiloxane, or a polymer that has no carbon-carbon bonds.

12. (canceled)

13. (canceled)

14. The vessel of claim 1, wherein the wall comprises an opening formed in the wall, and wherein the window is disposed in the opening.

15. The vessel of claim 14, wherein the window is configured to act as a gasket to seal the opening in the wall.

16. The vessel of claim 14, wherein the window seals the opening via at least one of compression of the polymer window, a press fit between the polymer window and the opening, lamination of the polymer window to the wall, thermal sealing, or adhering the window to the opening or the wall with an adhesive.

17. The vessel of claim 1, wherein the vessel is at least one of a flask, a multi-layer flask, a shaker flask, a spinner flask, a bioprocess bag, a bioreactor, a media bag, a bottle, a cryotube, and a piece of tubing.

18. The vessel of claim 17, wherein the vessel comprises tubing, and the window is disposed in at least one of a section of the tubing having a thinned wall, a fitting connected to or overmolded onto the tubing.

19. The vessel of claim 1, further comprising a retaining feature on the exterior of the vessel, wherein the retaining feature is configured to cooperate with the monitoring module to maintain the monitoring module in a position where the monitoring module can monitor the cell culture or the cell culture media through the window.

20. (canceled)

21. (canceled)

22. The vessel of claim 1, wherein the monitoring module comprises a probe with a distal end comprising a lens, wherein the probe is configured such that the lens faces the window when monitoring the cell culture chamber.

23. The vessel of claim 22, wherein the lens is in physical contact with the window when monitoring the cell culture chamber.

24-49. (canceled)

50. A method of monitoring a cell culture or cell culture environment, comprising:
providing a vessel according to claim 1;
providing a monitoring module comprising a measuring portion configured to measure an aspect of a cell culture or a cell culture environment, the measuring portion being disposed to measure the aspect in an interior of the vessel through the window of the vessel.

51-73. (canceled)
